(19) United States
(12) Patent Application Publication
(10) Pub. No.: US 2025/0129386 A1
(43) Pub. Date: Apr. 24, 2025

(54) NANOPARTICLE VECTOR FOR RNA SELF-DELIVERY, AND PREPARATION METHOD THEREFOR AND USE THEREOF

(71) Applicant: The Eighth Affiliated Hospital, Sun Yat-Sen University, Shenzehen (CN)

(72) Inventors: Meiling Zhu, Shenzhen (CN); Na Yang, Shenzhen (CN); Han Wang, Shenzhen (CN)

(21) Appl. No.: 18/539,295

(22) Filed: Dec. 14, 2023

(30) Foreign Application Priority Data
Oct. 18, 2023 (CN) 202311355319.1

(51) Int. Cl. C12N 15/88; A61K 47/32; A61K 47/40; A61K 47/69; C12N 15/113

(52) U.S. Cl. CPC C12N 15/88; A61K 47/32; A61K 47/40; A61K 47/6951; C12N 15/113; C12N 2310/14; C12N 2310/141; C12N 2310/351

(57) ABSTRACT

The present disclosure discloses a nanoparticle vector for RNA self-delivery and a preparation method therefor and use thereof. The nanoparticle vector includes: a β-cyclodextrin-RNA conjugate, an adamantane-ligand conjugate, and a cationic polymer, wherein the adamantane-ligand conjugate is formed by conjugating adamantane with a ligand molecule through polyethylene glycol. Through a host-guest interaction between β-cyclodextrin and an adamantane molecule, the nanoparticle vector can realize the modular conjugation of a delivered RNA molecule and the ligand molecule, and realize the self-delivery of RNA. The polyethylene glycol molecule can avoid the recognition and phagocytosis of immune cells before the vector enters target cells, and β-cyclodextrin can realize the escape of intracellular nucleosome. The nanoparticle vector delivers RNA to cells in a targeted manner, and is degraded in vivo via endocytosis, releasing RNA molecules to inhibit expression of genes of interest, and play a role in repairing cartilage and bone tissue in situ.

### CROSS-REFERENCE TO RELATED APPLICATION

The present application claims priority from Chinese Patent Application No. 202311355319.1 filed on Oct. 18, 2023, the contents of which are incorporated herein by reference in their entirety.

### REFERENCE TO SEQUENCE LISTING

This application includes a Sequence Listing filed electronically as a text file named Sequence Listing_XINYA-23017-USPT.xml, created on Dec. 11, 2023, with a size of 23 KB. The Sequence Listing is incorporated herein by reference.

### TECHNICAL FIELD

The present disclosure belongs to the technical field of biomedical materials, and in particular relates to a nanoparticle vector for RNA self-delivery, and a preparation method therefor and use thereof in bone tissue repair.

### BACKGROUND

Cartilage tissue is an important component of bone, is connective tissue which plays a supporting role, and is free of blood vessels and lymphatic vessels, and once the cartilage tissue is damaged, even minor cartilage damage, the cartilage tissue is difficult to repair or heal naturally. Therefore, regenerative repair of the cartilage tissue becomes one of the important research topics of current biomedicine. Currently, the commonly used method for cartilage tissue repair in the clinic is autologous cartilage transplantation, but this method has a risk that new trauma is caused to a patient and a certain pain is caused to the patient. To solve the above technical problem, the rapid development of cartilage tissue engineering provides a new approach for the treatment of articular cartilage diseases and defects, and the cartilage tissue engineering is to implant cartilage seeds into biodegradable and histocompatible biomaterials to form a composite, and then implant the composite into a cartilage defect, during the process of self-degradation of the biomaterials, the implanted cells form new cartilage to fill the defect, which requires three main conditions: (1) a sufficient number of normally functioning seed cells, (2) a suitable cell scaffold, and (3) cytokines that regulate cell proliferation and maintain cell phenotypic characteristics. However, the current scaffold system has the problems of poor targeting and large application limitations.

Studies have shown that RNA interference (RNAi) mediated by the delivery of gene vectors constructed from biomaterials has a certain regulatory effect on gene expression in cells, which has a role in enhancing the therapeutic effect of diseases (Madry H, Gao L, Rey-Rico A, et al. Thermosensitive hydrogel based on PEO-PPO-PEO poloxamers for a controlled in situ release of recombinant adeno-associated viral vectors for effective gene therapy of cartilage defects [J]. Advanced Materials, 2020, 32 (2): 1906508.), and has important application values for investigating complex biological processes in vivo (Dahlman J E, Barnes C, Khan O F, et al. In vivo endothelial siRNA delivery using polymeric nanoparticles with low molecular weight [J]. Nature nanotechnology, 2014, 9(8): 648-655.). Compared with traditional scaffold systems for cartilage tissue repair treatment, there are more precise therapeutic targets, and corresponding nucleic acid types and structures can be designed according to the corresponding targets (Zhu M, Wei K, Lin S, et al. Bioadhesive polymersome for localized and sustained drug delivery at pathological sites with harsh enzymatic and fluidic environment via supramolecular host-guest complexation [J]. Small, 2018, 14 (7): 1702288.). In such vectors, small interfering RNA (siRNA) and small molecule RNA (MicroRNA, miRNA) are electronegative oligonucleotide molecules, and a nucleic acid structure of RNA can be chemically modified or RNA can be made suitable for conduction and tissue repair in vivo by using an inhibitor (Alterman J F, Godinho B M D C, Hassler M R, et al. A divalent siRNA chemical scaffold for potent and sustained modulation of gene expression throughout the central nervous system [J]. Nature biotechnology, 2019, 37 (8): 884-894.). Functionalized siRNA as well as antisense oligonucleotides (ASOs) have been proved to be useful in the treatment of liver diseases, and recently, researchers have also conducted research on their use in other organ tissue diseases, such as for the treatment of bone, cartilage tissue and central nervous system diseases (Finkel R S, Mercuri E, Darras B T, et al. Nusinersen versus sham control in infantile-onset spinal muscular atrophy [J]. N Engl J Med, 2017, 377: 1723-1732. Yang R, Chen F, Guo J, et al. Recent advances in polymeric biomaterials-based gene delivery for cartilage repair [J]. Bioactive Materials, 2020, 5(4): 990-1003. Rajeev K G, Nair J K, Jayaraman M, et al. Hepatocyte-specific delivery of siRNAs conjugated to novel non-nucleosidic trivalent N-acetylgalactosamine elicits robust gene silencing in vivo [J]. ChemBioChem, 2015, 16(6): 903-908.).

In addition, in recent years, various subtypes of biomaterials have been developed as vectors for gene therapy (Badieyan Z S, Berezhanskyy T, Utzinger M, et al. Transcript-activated collagen matrix as sustained mRNA delivery system for bone regeneration [J]. Journal of Controlled Release, 2016, 239: 137-148. Kaneti L, Bronshtein T, Malkah Dayan N, et al. Nanoghosts as a novel natural nonviral gene delivery platform safely targeting multiple cancers [J]. Nano letters, 2016, 16 (3): 1574-1582.), while due to the wide variety of polymer materials, the application of modified polymer materials in gene vectors and scaffold materials has objective prospects (Yang R, Chen F, Guo J, et al. Recent advances in polymeric biomaterials-based gene delivery for cartilage repair [J]. Bioactive Materials, 2020, 5(4): 990-1003.). Currently, gene therapy based on siRNA and microRNA is mainly hindered by two biological barriers of the body during application: extracellular and intracellular barriers (Lorenzer C, Dirin M, Winkler A M, et al. Going beyond the liver: progress and challenges of targeted delivery of siRNA therapeutics [J]. Journal of Controlled Release, 2015, 203: 1-15. Cavallaro G, Sardo C, Craparo E F, et al. Polymeric nanoparticles for siRNA delivery: Production and applications [J]. International journal of pharmaceutics, 2017, 525 (2): 313-333.). RNA-loaded vectors have been metabolized out by the glomerular filtration of the body and enzymatic degradation, or are unlikely to reach target cells and exert their effects due to lack of targeting (Cavallaro G, Sardo C, Craparo E F, et al. Polymeric nanoparticles for siRNA delivery: Production and applications [J]. International journal of pharmaceutics, 2017, 525 (2): 313-333.), and polymer materials can be modified by different methods to obtain corresponding functions, so that scaffolds and vector materials can play a more efficient role in targeted delivery and gene regulation (Wang D, Lin J, Jia F, et al. Bottlebrush-architectured poly(ethylene glycol) as an efficient vector for RNA interference in vivo [J]. Science advances, 2019, 5(2): eaav9322.). In the field of cartilage tissue repair, some research achievements have been made in the preparation of biological scaffold composites by using biomaterials. However, in previous studies, most of the scaffold materials have fixed scaffold structures (Zhang X, Li Y, Chen Y E, et al. Cell-free 3D scaffold with two-stage delivery of miRNA-26a to regenerate critical-sized bone defects [J]. Nature communications, 2016, 7 (1): 1-15.), or viruses are used as gene vectors, which has great application limitations. At the same time, some by-products are inevitably introduced in the process of introducing chemical components (Malhotra M, Gooding M, Evans J C, et al. Cyclodextrin-siRNA conjugates as versatile gene silencing agents [J]. European Journal of Pharmaceutical Sciences, 2018, 114: 30-37.), which leads to certain cytotoxicity, or the application of the vectors is limited due to the specificity of the structure and composition (Cavallaro G, Sardo C, Craparo E F, et al. Polymeric nanoparticles for siRNA delivery: Production and applications [J]. International journal of pharmaceutics, 2017, 525 (2): 313-333.).

In view of this, providing anew biomedical material for bone tissue repair has become an urgent technical problem to be solved.

### SUMMARY

Therefore, the technical problem to be solved by the present disclosure is that the conventional bone tissue repair material has a large application limitation and toxic and side effects, thereby providing a nanoparticle vector for RNA self-delivery having a wider application range and no toxic and side effects, and a preparation method therefor and use thereof in bone tissue repair.

To solve the above technical problems, the technical solutions of the present disclosure are as follows:
in a first aspect, the present disclosure provides a nanoparticle vector for RNA self-delivery, including a β-cyclodextrin-RNA conjugate, an adamantane-ligand conjugate and a cationic polymer, wherein the adamantane-ligand conjugate is formed by conjugating adamantane with a ligand molecule through polyethylene glycol, and a molar ratio of the β-cyclodextrin-RNA conjugate to the adamantane-ligand conjugate to the cationic polymer is 1:1:1-200.

Preferably, the RNA is siRNA or microRNA; and the ligand is CXCR4 or a cell penetrating peptide.

Preferably, the nanoparticle vector for RNA self-delivery has an average particle size of not greater than 200 nm.

In a second aspect, the present disclosure provides a preparation method for the nanoparticle vector for RNA self-delivery, including the steps of:
S1, preparation of a β-cyclodextrin-RNA conjugate:
connecting a linking group for linking β-cyclodextrin at an end of an RNA molecule to obtain an RNA molecule having the linking group; and
reacting β-cyclodextrin with an active maleimide molecule, carrying out an equimolar reaction between β-cyclodextrin and the RNA molecule having the linking group by Michael addition, and connecting the RNA molecule having the linking group to a sugar ring side group of the β-cyclodextrin;
S2, preparation of an adamantane-ligand conjugate:
reacting a polyethylene glycol (PEG) molecule with adamantane to obtain an adamantane ligand molecule: ADA-PEG-ligand, and
reacting a ligand with an equimolar amount of the adamantane ligand molecule to obtain the adamantane-ligand conjugate;
S3, carrying out an equimolar reaction between the β-cyclodextrin-RNA conjugate obtained in the step S1 and the adamantane-ligand conjugate obtained in the step S2 to obtain a β-cyclodextrin-RNA-adamantane-ligand conjugation complex; and
S4, preparing the nanoparticle vector for RNA self-delivery by reacting a cationic polymer with the conjugation complex obtained in the step S3 by using a nanosedimentation method, wherein a molar ratio of the conjugation complex to the cationic polymer is 1:1-200.

Preferably, the RNA is siRNA or microRNA, and in the step S1, connecting the linking group for linking β-cyclodextrin at the end of the RNA molecule includes modifying sulfhydryl at a 5′ end of a sense strand of a double-stranded molecule of siRNA, or modifying sulfhydryl at an end of a single-stranded molecule of microRNA.

Preferably, the step S1 further includes a step of modifying a fluorescent group at a 3′ end of the sense strand of the double-stranded molecule of siRNA, the fluorescent group being a red fluorescent group.

Preferably, the polyethylene glycol is at least one of PEG MW 500, PEG MW 2500, and PEG MW 5000.

Preferably, the β-cyclodextrin is mono(6-amino-6-deoxy)-3-cyclodextrin: NH₂-β-CD.

In a third aspect, the present disclosure provides use of the nanoparticle vector for RNA self-delivery for loading bone tissue repair drugs.

Preferably, the nanoparticle vector for RNA self-delivery may be prepared as an injectable gel including the nanoparticle vector, and further including a hyaluronic acid-cyclodextrin macromer, the nanoparticle vector and the hyaluronic acid-cyclodextrin macromer being linked by a linker molecule ADA-PEG-ADA terminated with an adamantane molecule to form a gel complex.

Compared with the prior art, the above technical solutions of the present disclosure have the following advantages:
the nanoparticle vector for RNA self-delivery provided by the present disclosure includes: the β-cyclodextrin-RNA conjugate, the adamantane-ligand conjugate, and the cationic polymer, wherein the adamantane-ligand conjugate is formed by conjugating adamantane with the ligand molecule through polyethylene glycol. Through a host-guest interaction between β-cyclodextrin and the adamantane molecule, the nanoparticle vector can realize the modular conjugation of a delivered RNA molecule and the ligand molecule, and realize the self-delivery of RNA. At the same time, adamantane and the ligand molecule are connected through a polyethylene glycol molecule, the polyethylene glycol molecule can avoid the recognition and phagocytosis of immune cells before the vector enters target cells, and β-cyclodextrin can realize the escape of intracellular nucleosome, thus better realizing the function of gene silencing. The nanoparticle vector delivers RNA to cells in a targeted manner, and is degraded in vivo via endocytosis, releasing RNA molecules to inhibit expression of genes of interest, and play a role in repairing cartilage and bone tissue in situ. The nanoparticle vector for RNA self-delivery provided by the present disclosure is suitable for loading bone tissue repair drugs.

### BRIEF DESCRIPTION OF FIGURES

In order to make the contents of the present disclosure easier to clearly understood, the present disclosure will be further described below in detail with reference to specific embodiments of the present disclosure and the accompanying drawings, wherein:

FIG. 1 is a schematic diagram of the formation and action of a nanoparticle vector for RNA self-delivery provided by an example of the present disclosure;

FIG. 2 is an extended application of a nanoparticle vector for RNA self-delivery provided by an example of the present disclosure: a schematic diagram of the formation and action of an injectable gel;
(a) in FIGS. 3 and (b) in FIG. 3 are SEM images of a nanoparticle vector for RNA self-delivery provided in Example 1 of the present disclosure;

FIG. 4 is a laser confocal microscopic image of human bone marrow mesenchymal stem cells before and after co-culture with the nanoparticle vector for RNA self-delivery provided in Example 1; and

FIG. 5: (a) is a cell viability test diagram of the nanoparticle vector for RNA self-delivery provided in Example 1; (b) is a comparison diagram of transfection effects of the nanoparticle vector for RNA self-delivery provided in Example 1 and a control group; and (c) is a comparison diagram of fluorescent labeling effects of the nanoparticle vector for RNA self-delivery provided in Example 1 and the control group.

### DETAILED DESCRIPTION

### Example 1

This example provides a nanoparticle vector for RNA self-delivery, including: a β-cyclodextrin-RNA conjugate, an adamantane-ligand conjugate, and a cationic polymer, wherein the β-cyclodextrin-RNA conjugate is formed by covalent binding of β-cyclodextrin to targeted siRNA, and the adamantane-ligand conjugate is formed by linking adamantane to a ligand molecule through polyethylene glycol. In this example, a molar ratio of the β-cyclodextrin-RNA conjugate to the adamantane-ligand conjugate to the cationic polymer is 1:1:50, the ligand is a cell penetrating peptide, and the cationic polymer is polyethyleneimine (PEI).

The nanoparticle vector for RNA self-delivery provided in this example was prepared by the following method:
S1, preparation of a β-cyclodextrin-RNA (siEGR1) conjugate:
a double-stranded small interfering RNA molecule siEGR1 (which includes a sense strand and an antisense strand) was designed with mRNA translating EGR1 as a target. Sulfhydryl (-SH) was connected at a 5′ end of the sense strand for connection to β-cyclodextrin to obtain an RNA molecule with a linking group; a red fluorescent group was connected at a 3′ end of the sense strand, and the 3′ and 5′ ends of the antisense strand were retained to retain the activity of RNA; wherein a sequence of siEGR1 is:

Sequence 1:
hs-EGR1-si-1:
(SEQ ID NO: 1)
sense (5′-3′)AGAUGAUGCUGCUGAGCAAdTdT,
(SEQ ID NO: 2)
antisense (5′-3′)UUGCUCAGCAGCAUCAUCUdTdT.
Sequence 2:
hs-EGR1-si-2:
(SEQ ID NO: 3)
sense (5′-3′)GCAGCAGCAGCACCUUCAAdTdT,
(SEQ ID NO: 4)
antisense (5′-3′)UUGAAGGUGCUGCUGCUGCdTdT.
Sequence 3:
hs-EGR1-si-3:
(SEQ ID NO: 5)
sense (5′-3′)GGACAAGAAAGCAGACAAAdTdT,
(SEQ ID NO: 6)
antisense (5′-3′)UUUGUCUGCUUUCUUGUCCdTdT.

β-cyclodextrin containing an amino side group (mono(6-amino-6-deoxy)-β-cyclodextrin/NH₂-β-CD, having a chemical formula of C₄₂H₇₁NO₃₄, and a molecular weight of 1134, Shandong Binzhou Zhiyuan Biotechnology Co., Ltd.) reacted with active maleimide to obtain a reaction product, and the above reaction product reacted with an RNA molecule connected with sulfhydryl (-SH) and a red fluorescent group (SH-siEGR1) in an equimolar amount by Michael addition to connect the RNA molecule terminated with sulfhydryl to a sugar ring side group of β-cyclodextrin.
S2, Preparation of an adamantane-ligand conjugate:
polyethylene glycols (PEGs) of different specifications were selected to react with adamantane to obtain adamantane-polyethylene glycol products. In this example, the polyethylene glycols used were PEG MW 500, PEG MW 2500, and PEG MW 5000.

A cell penetrating peptide TAT was used as a ligand to react with an equimolar amount of the adamantane-polyethylene glycol product to connect the cell penetrating peptide to the end of the adamantane-polyethylene glycol molecule to obtain the adamantane-ligand conjugate (ADA-PEG-TAT).
S3, The adamantane-ligand conjugate reacted with the β-cyclodextrin-RNA conjugate obtained in the step S1 in an equimolar amount to obtain a conjugation complex: a β-cyclodextrin-RNA-adamantane-ligand conjugation complex by a host-guest interaction between β-cyclodextrin and the adamantane molecule.
S4, The nanoparticle vector for RNA self-delivery was prepared by reacting polyethyleneimine with the conjugation complex obtained in the step S3 by using a nanosedimentation method, wherein nanoparticles were self-assembled by using electrostatic adsorption of RNA and the cationic polymer, and in this example, a molar ratio of the conjugation complex to polyethyleneimine was 1:50.

The nanoparticle vector for RNA self-delivery provided in this example has an average particle size of not greater than 200 nm.

According to the nanoparticle vector for RNA self-delivery provided in this example, on one hand, because the encapsulated RNA molecules also participate in the nanoparticle self-assembly process during preparation, thereby avoiding the excessive introduction of by-products within the system, achieving self-assembly of RNA; on the other hand, adamantane is linked to the ligand molecule through polyethylene glycol, polyethylene glycol may avoid recognition and phagocytosis of immune cells before the vector enters target cells; and in addition, β-cyclodextrin contains an amino side group, which is equivalent to the addition of an “easily protonated amino group” in the vector, which can achieve the escape of intracellular nucleosome, thereby forming an induced silencing complex, and better realizing the function of gene silencing.

The nanoparticle vector for RNA self-delivery provided in this example can deliver siEGR1 to cells in a targeted manner, and is degraded in vivo via endocytosis, releasing siEGR1 to inhibit expression of an EGR1 gene.

### Example 2

This example provides a nanoparticle vector for RNA self-delivery, including: a β-cyclodextrin-RNA conjugate, an adamantane-ligand conjugate, and a cationic polymer, wherein the β-cyclodextrin-RNA conjugate is formed by covalent binding of β-cyclodextrin to targeted siRNA, and the adamantane-ligand conjugate is formed by linking adamantane to a ligand molecule through polyethylene glycol. In this example, a molar ratio of the β-cyclodextrin-RNA conjugate to the adamantane-ligand conjugate to the cationic polymer is 1:1:180, the ligand is CXCR4 (a chemokine receptor), and the cationic polymer is polyethyleneimine (PEI).

The nanoparticle vector for RNA self-delivery provided in this example was prepared by the following method:
S1, preparation of a β-cyclodextrin-RNA (siRNA) conjugate:
a double-stranded small interfering RNA molecule siColX (which includes a sense strand and an antisense strand) was designed with ColX as a target. Sulfhydryl (-SH) was connected at a 5′ end of the sense strand for connection to β-cyclodextrin to obtain an RNA molecule with a linking group; a red fluorescent group was connected at a 3′ end of the sense strand, and the 3′ and 5′ ends of the antisense strand were retained to retain the activity of RNA; wherein a sequence of siCOL10A1 is:

Sequence 1:
hs-COL10A1-si-1:
(SEQ ID NO: 7)
sense (5′-3′)CCCUACACCAUAAAGAGUAAAdTdT,
(SEQ ID NO: 8)
antisense (5′-3′)UUUACUCUUUAUGGUGUAGGGdTdT.
Sequence 2:
hs-COL10A1-si-2:
(SEQ ID NO: 9)
sense (5′-3′)CCUGUAAUGUACACCUAUGAUdTdT,
(SEQ ID NO: 10)
antisense (5′-3′)AUCAUAGGUGUACAUUACAGGdTdT.
Sequence 3:
hs-COL10A1-si-3:
(SEQ ID NO: 11)
sense (5′-3′)GCAUCAAAGGUGAUAGAGGUUdTdT,
(SEQ ID NO: 12)
antisense (5′-3′)AACCUCUAUCACCUUUGAUGCdTdT.

β-cyclodextrin containing an amino side group (mono(6-amino-6-deoxy)-β-cyclodextrin/NH₂-β-CD, having a chemical formula of C₄₂H₇NO₃₄, and a molecular weight of 1134, Shandong Binzhou Zhiyuan Biotechnology Co., Ltd.) reacted with active maleimide to obtain a reaction product, and the above reaction product reacted with an RNA molecule connected with sulfhydryl (-SH) and a red fluorescent group (SH-siColX) in an equimolar amount by Michael addition to connect the RNA molecule terminated with sulfhydryl to a sugar ring side group of β-cyclodextrin.
S2, Preparation of an adamantane-ligand conjugate:
polyethylene glycols (PEGs) of different specifications were selected to react with adamantane to obtain adamantane-polyethylene glycol products. In this example, the polyethylene glycols used were PEG MW 500, PEG MW 2500, and PEG MW 5000.

CXCR4 was used as a ligand to react with an equimolar amount of the adamantane-polyethylene glycol product to connect the ligand CXCR4 to the end of the adamantane-polyethylene glycol molecule to obtain the adamantane-ligand conjugate (ADA-PEG-CXCR4).
S3, The adamantane-ligand conjugate reacted with the β-cyclodextrin-RNA conjugate obtained in the step S1 in an equimolar amount to obtain a conjugation complex: a β-cyclodextrin-RNA-adamantane-ligand conjugation complex by a host-guest interaction between β-cyclodextrin and the adamantane molecule.
S4, The nanoparticle vector for RNA self-delivery was prepared by reacting polyethyleneimine with the conjugation complex obtained in the step S3 by using a nanosedimentation method, wherein nanoparticles were self-assembled by using electrostatic adsorption of RNA and the cationic polymer, and in this example, a molar ratio of the conjugation complex to polyethyleneimine was 1:180.

The nanoparticle vector for RNA self-delivery provided in this example has an average particle size of not greater than 200 nm.

According to the nanoparticle vector for RNA self-delivery provided in this example, on one hand, because the encapsulated RNA molecules also participate in the nanoparticle self-assembly process during preparation, thereby avoiding the excessive introduction of by-products within the system, achieving self-assembly of RNA; on the other hand, adamantane is linked to the ligand molecule through polyethylene glycol, polyethylene glycol may avoid recognition and phagocytosis of immune cells before the vector enters target cells; and in addition, β-cyclodextrin contains an amino side group, which is equivalent to the addition of an “easily protonated amino group” in the vector, which can achieve the escape of intracellular nucleosome, thereby forming an induced silencing complex, and better realizing the function of gene silencing.

The nanoparticle vector for RNA self-delivery provided in this example delivers siColX which can inhibit the hypertrophy of mesenchymal stem cells hMSCs and promote chondrogenic differentiation of the mesenchymal stem cells to cells in a targeted manner, is degraded in vivo by endocytosis of the cells, releasing siColX, thereby achieving biomaterial-mediated targeted delivery of gene vectors, achieving the in-situ repair function of articular cartilage tissue through the promotion of chondrogenic differentiation of hMSCs by siColX, thereby obtaining a material suitable for loading in-situ cartilage tissue repair drugs, and making the nanoparticle vector for RNA self-delivery provided by the present disclosure suitable for loading bone tissue repair drugs.

### Example 3

This example provides a nanoparticle vector for RNA self-delivery, including: a β-cyclodextrin-RNA conjugate, an adamantane-ligand conjugate, and a cationic polymer, wherein the β-cyclodextrin-RNA conjugate is formed by covalent binding of β-cyclodextrin to targeted microRNA, and the adamantane-ligand conjugate is formed by linking adamantane to a ligand molecule through polyethylene glycol. In this example, a molar ratio of the β-cyclodextrin-RNA conjugate to the adamantane-ligand conjugate to the cationic polymer is 1:1:200, the ligand is CXCR4 (a chemokine receptor), and the cationic polymer is polyethyleneimine (PEI).

The nanoparticle vector for RNA self-delivery provided in this example was prepared by the following method:
S1, preparation of a β-cyclodextrin-RNA (microRNA) conjugate:
a microRNA molecule, Mir218, was first designed, and modified with sulfhydryl (-SH) for connection to β-cyclodextrin to obtain an RNA molecule with a linking group. A sequence of Mir218 is:

Sequence 1:
(SEQ ID NO: 13)
hsa-miR-218-5p UUGUGCUUGAUCUAACCAUGU.
Sequence 2:
(SEQ ID NO: 14)
hsa-miR-218-1-3p AUGGUUCCGUCAAGCACCAUGG.

β-cyclodextrin containing an amino side group (mono(6-amino-6-deoxy)-β-cyclodextrin/NH₂-β-CD, having a chemical formula of C₄₂H₇₁NO₃₄, and a molecular weight of 1134, Shandong Binzhou Zhiyuan Biotechnology Co., Ltd.) reacted with active maleimide to obtain a reaction product, and the above reaction product reacted with an RNA molecule connected with sulfhydryl (-SH) (SH-mir218) in an equimolar amount by Michael addition to connect the RNA molecule terminated with sulfhydryl to a sugar ring side group of β-cyclodextrin.
S2, preparation of an adamantane-ligand conjugate:
polyethylene glycols (PEGs) of different specifications were selected to react with adamantane to obtain adamantane-polyethylene glycol products. In this example, the polyethylene glycols used were PEG MW 500, PEG MW 2500, and PEG MW 5000.

CXCR4 was used as a ligand to react with an equimolar amount of the adamantane-polyethylene glycol product to connect the ligand molecule CXCR4 to the end of the adamantane-polyethylene glycol molecule to obtain the adamantane-ligand conjugate (ADA-PEG-CXCR4).
S3, The adamantane-ligand conjugate reacted with the β-cyclodextrin-RNA conjugate obtained in the step S1 in an equimolar amount to obtain a conjugation complex: a β-cyclodextrin-RNA-adamantane-ligand conjugation complex by a host-guest interaction between β-cyclodextrin and the adamantane molecule.
S4, The nanoparticle vector for RNA self-delivery was prepared by reacting polyethyleneimine with the conjugation complex obtained in the step S3 by using a nanosedimentation method, wherein nanoparticles were self-assembled by using electrostatic adsorption of RNA and the cationic polymer, and in this example, a molar ratio of the conjugation complex to polyethyleneimine was 1:200.

The nanoparticle vector for RNA self-delivery provided in this example has an average particle size of not greater than 200 nm.

According to the nanoparticle vector for RNA self-delivery provided in this example, on one hand, because the encapsulated RNA molecules also participate in the nanoparticle self-assembly process during preparation, thereby avoiding the excessive introduction of by-products within the system, achieving self-assembly of RNA; on the other hand, adamantane is linked to the ligand molecule through polyethylene glycol, polyethylene glycol may avoid recognition and phagocytosis of immune cells before the vector enters target cells; and in addition, β-cyclodextrin contains an amino side group, which is equivalent to the addition of an “easily protonated amino group” in the vector, which can achieve the escape of intracellular nucleosome, thereby forming an induced silencing complex, and better realizing the function of gene silencing.

The nanoparticle vector for RNA self-delivery provided in this example delivers microRNA mir218 which can promote the endochondral osteogenic differentiation of hMSCs to cells in a targeted manner, is degraded in vivo by endocytosis of the cells, releasing mir218, thereby achieving biomaterial-mediated targeted delivery of gene vectors, achieving the in-situ repair effect of bone tissue through the promotion of endochondral osteogenic differentiation of hMSCs by mir218, thereby obtaining a material suitable for loading in-situ bone tissue repair drugs, and making the nanoparticle vector for RNA self-delivery provided by the present disclosure suitable for loading bone tissue repair drugs.

### Experimental Example

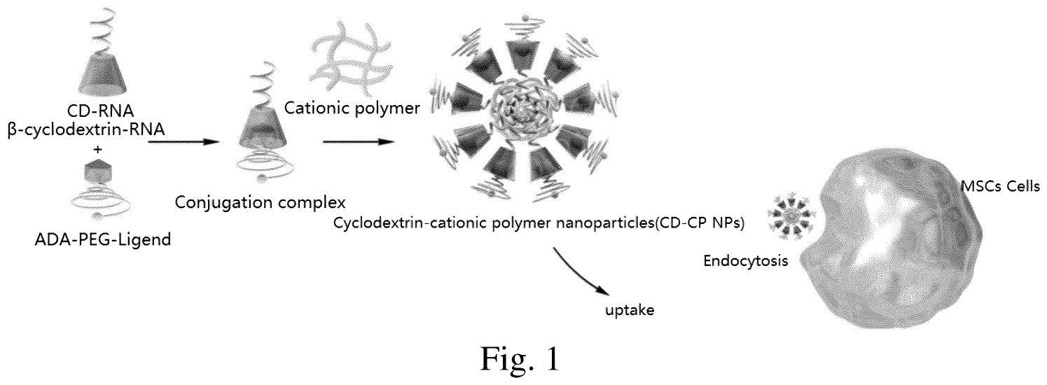

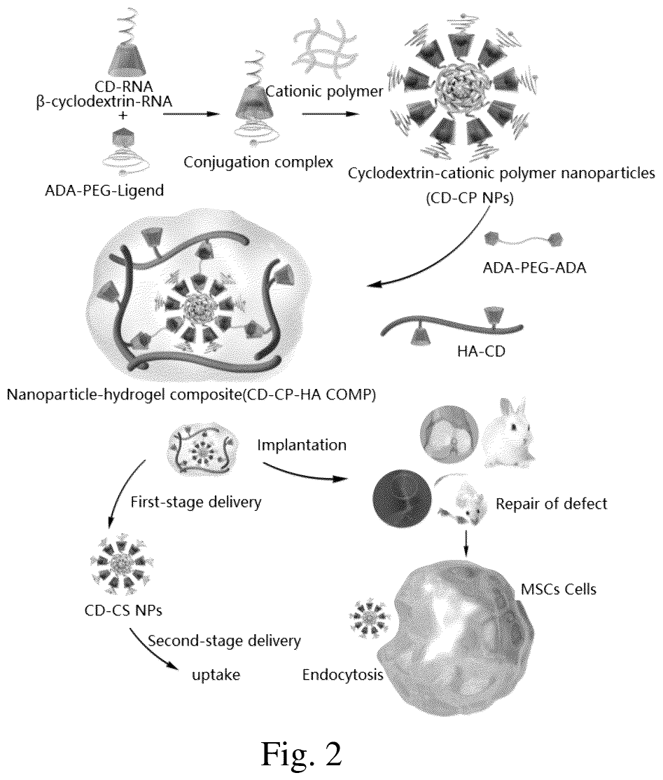

1. A schematic diagram of the formation and action of the nanoparticle vectors for RNA self-delivery provided in the examples of the present application is shown in FIG. 1.

As can be seen in the figure, the nanoparticle vector for RNA self-delivery is formed based on modular conjugation of ligands, and the nanoparticle vector delivers RNA molecules to cells in a targeted manner, and is degraded in vivo by endocytosis of cells, releasing RNA molecules to inhibit expression of genes of interest, achieving in situ repair of cartilage and bone tissue.

2. A schematic diagram of the formation and action of an injectable gel using the nanoparticle vector for RNA self-delivery provided in the examples of the present application is shown in FIG. 2.

A hyaluronic acid physical gel complex (CD-CP-HA COMP) with the nanoparticle vector as a crosslinking point and hyaluronic acid-cyclodextrin as a macromer was constructed by introducing a linker molecule (ADA-PEG-ADA), namely an adamantane molecule-terminated polyethylene glycol molecule. The gel composite can be injected at a site of a tissue defect for in situ repair of the tissue by two-stage drug delivery.

3. SEM images of the nanoparticle vector for RNA self-delivery provided in Example 1 were tested, and the test results are shown in (a) and (b) in FIG. 3, wherein (a) in FIG. 3 shows a morphology of the nanoparticle vector under a low power lens and (b) in FIG. 3 shows a morphology of the nanoparticle vector under a high power lens, and it can be seen from the figures that the nanoparticle vector has a morphologically regular spherical shape with a particle size within 200 nm.

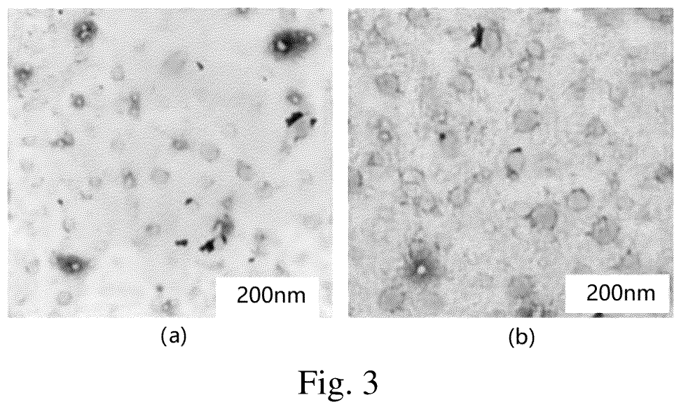

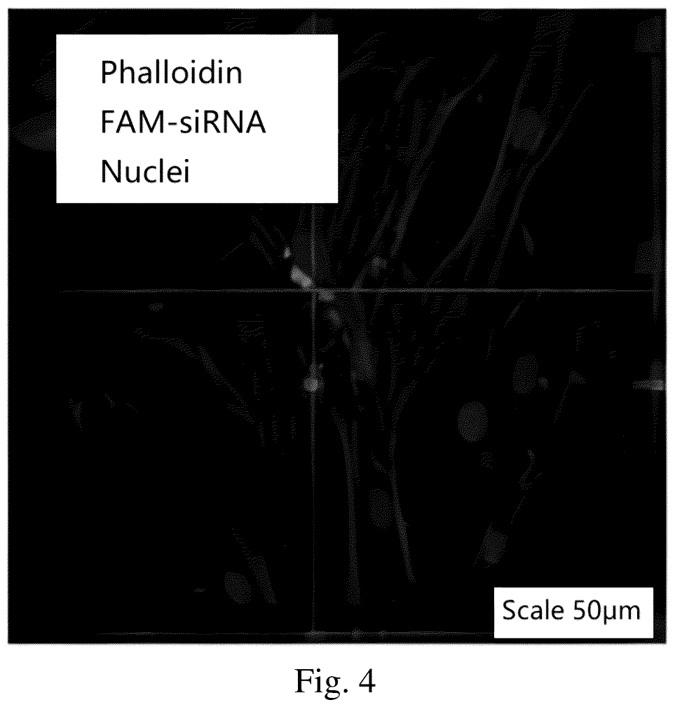

4. Human bone marrow mesenchymal stem cells (hMSCs) were cultured in serum-free starvation for 4 h, and the FAM-siRNA-encapsulated CD-CP NPs provided in Example 1 and the mesenchymal stem cells hMSCs were co-cultured for 24 hours, and subjected to fixed staining to test a laser confocal microscopic image. The test results are shown in FIG. 4.

The culture conditions were as follows: an α-MEM medium containing 16.7% fetal bovine serum, 1% L-glutamine and 1% P/S was adopted.

Green fluorescence can be seen from the figure, indicating that the prepared nanoparticle vector can efficiently transfect small interfering RNA into the mesenchymal stem cells hMSCs. Where, red: a phalloidin stained cytoskeletal structure, green: FITC dye or FAM green fluorescence, and blue: a DAPI stained nuclear structure.

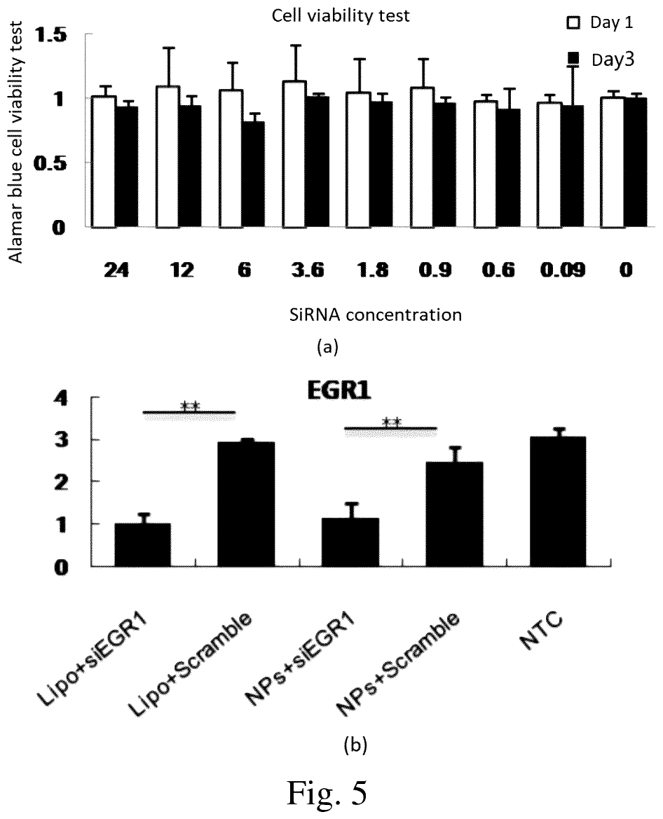

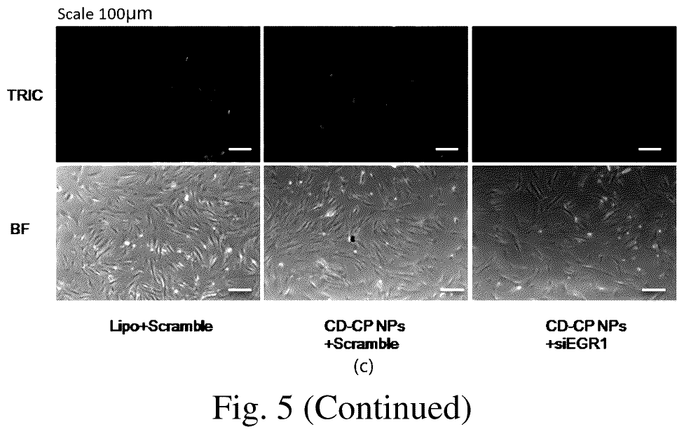

5. A nanoparticle vector for RNA self-delivery in a control group was prepared by using siRNA (scramble) with equally modified conditions by the preparation method provided in Example 1. The cell viability data of the nanoparticle vector for RNA self-delivery provided in Example 1, comparison of the inhibitory effects of an siEGR1 gene and a control gene (scramble) which were encapsulated in the nanoparticle vector provided in Example 1 with those of siEGR1 transfected with a commercial transfection reagent lipo3000 on EGR1 gene expression, and comparison of the fluorescence labeling effects of the siEGR1 gene and the control gene which were transfected with the nanoparticle vector provided in Example 1 for 24 hours were tested; and the test results are shown in (a)-(c) in FIG. 5.

As can be seen from (a) in FIG. 5, by co-culturing cyclodextrin-polyethylenimine nanoparticle vectors encapsulating a gradient concentration (0 to 24 nM) of small interfering RNA with mesenchymal stem cells hMSCs (specific culturing steps were as follows: after culture in serum-free starvation for 4 h, nanoparticle vectors encapsulating a gradient concentration of siRNA were co-cultured with the mesenchymal stem cells hMSCs for 24 hours), cell viability test data (day 1 and day 3) measured by Alamar blue staining demonstrated good cytocompatibility of the nanoparticle vector for RNA self-delivery in short-term culture.

As can be seen from (b) in FIG. 5, the inhibitory effect of the siEGR1 gene-encapsulated nanoparticle vector provided in Example 1 on EGR1 gene expression is comparable to that of commercial reagents.

As can be seen from (c) in FIG. 5, TRIC of the CD-CP NPs+siEGR1 group provided in Example 1 was substantially non-fluorescent compared with the control group (CD-CP NPs+Scramble), indicating that the prepared siEGR1 gene-loaded cyclodextrin-polyethyleneimine nanoparticle vectors (CD-PEI NPs) can efficiently transfect siEGR1 siRNA into the mesenchymal stem cells (hMSCs), and that siEGR1 siRNA exerts gene silencing effects. TRIC of both the Lipo+Scramble group and the CD-CP NPs+Scramble group showed bright red fluorescence, indicating that CD-CD-CP NPs had no effect on the gene silencing effect of siEGR1 siRNA, while a negative control group was set to reversedly demonstrate that siEGR1 can be efficiently transfected into cells by the nanoparticle vector CD-PEI NPs and exert its effects. (**P<0.01, n=3).

Obviously, the above examples are instances only for clear description, rather than limiting the embodiments. For those of ordinary skill in the art, other different forms of variations or changes can be made on the basis of the above description. It is unnecessary and impossible to enumerate all the embodiments here. Obvious variations or changes derived therefrom are still within the scope of protection of the present disclosure.

What is claimed is:

1. A nanoparticle vector for RNA self-delivery, comprising a β-cyclodextrin-RNA conjugate, an adamantane-ligand conjugate, and a cationic polymer, wherein the adamantane-ligand conjugate is formed by conjugating adamantane with a ligand molecule through polyethylene glycol, and a molar ratio of the β-cyclodextrin-RNA conjugate to the adamantane-ligand conjugate to the cationic polymer is 1:1:1-200.

2. The nanoparticle vector for RNA self-delivery according to claim 1, wherein the RNA is siRNA or microRNA; and the ligand is CXCR4 or a cell penetrating peptide.

3. The nanoparticle vector for RNA self-delivery according to claim 2, wherein the nanoparticle vector for RNA self-delivery has an average particle size of not greater than 200 nm.

4. A preparation method for the nanoparticle vector for RNA self-delivery according to any claim 1, comprising the steps of:
S1, preparation of a β-cyclodextrin-RNA conjugate:
connecting a linking group for linking β-cyclodextrin at an end of an RNA molecule to obtain an RNA molecule having the linking group; and
reacting β-cyclodextrin with an active maleimide molecule, carrying out an equimolar reaction between β-cyclodextrin and the RNA molecule having the linking group by Michael addition, and connecting the RNA molecule having the linking group to a sugar ring side group of the β-cyclodextrin;
S2, preparation of an adamantane-ligand conjugate:
reacting a polyethylene glycol (PEG) molecule with adamantane to obtain an adamantane ligand molecule: ADA-PEG-ligand, and
reacting a ligand with an equimolar amount of the adamantane ligand molecule to obtain the adamantane-ligand conjugate;
S3, carrying out an equimolar reaction between the β-cyclodextrin-RNA conjugate obtained in the step S1 and the adamantane-ligand conjugate obtained in the step S2 to obtain a β-cyclodextrin-RNA-adamantane-ligand conjugation complex; and
S4, preparing the nanoparticle vector for RNA self-delivery by reacting a cationic polymer with the conjugation complex obtained in the step S3 by using a nanosedimentation method, wherein a molar ratio of the conjugation complex to the cationic polymer is 1:1-200.

5. The preparation method according to claim 4, wherein the RNA is siRNA or microRNA, and in the step S1, connecting the linking group for linking β-cyclodextrin at the end of the RNA molecule comprises modifying sulfhydryl at a 5′ end of a sense strand of a double-stranded molecule of siRNA, or modifying sulfhydryl at an end of a single-stranded molecule of microRNA.

6. The preparation method according to claim 5, wherein the step S1 further comprises a step of modifying a fluorescent group at a 3′ end of the sense strand of the double-stranded molecule of siRNA, the fluorescent group being a red fluorescent group.

7. The preparation method according to claim 4, wherein the polyethylene glycol is at least one of PEG MW 500, PEG MW 2500, and PEG MW 5000.

8. The preparation method according to claim 4, wherein the β-cyclodextrin is mono(6-amino-6-deoxy)-β-cyclodextrin: NH₂-β-CD.

9. Use of the nanoparticle vector for RNA self-delivery according to claim 1 for loading bone tissue repair drugs.

10. The use according to claim 9, wherein the nanoparticle vector for RNA self-delivery can be prepared as an injectable gel comprising the nanoparticle vector, and further comprising a hyaluronic acid-cyclodextrin macromer, the nanoparticle vector and the hyaluronic acid-cyclodextrin macromer being linked by a linker molecule ADA-PEG-ADA terminated with an adamantane molecule to form a gel complex.


SEQUENCE LISTING (14 sequences)

SEQ ID NO: 1 - RNA, 21 nt, synthetic construct
  source 1..21: mol_type = other RNA
  misc_feature 1..21: note = hs-EGR1-si-1 sense strand
  misc_feature 20..21: note = n=dT
agatgatgctgctgagcaann

SEQ ID NO: 2 - RNA, 21 nt, synthetic construct
  source 1..21: mol_type = other RNA
  misc_feature 1..21: note = hs-EGR1-si-1 antisense strand
  misc_feature 20..21: note = n=dT
ttgctcagcagcatcatctnn

SEQ ID NO: 3 - RNA, 21 nt, synthetic construct
  source 1..21: mol_type = other RNA
  misc_feature 1..21: note = hs-EGR1-si-2 sense strand
  misc_feature 20..21: note = n=dT
gcagcagcagcaccttcaann

SEQ ID NO: 4 - RNA, 21 nt, synthetic construct
  source 1..21: mol_type = other RNA
  misc_feature 1..21: note = hs-EGR1-si-2 antisense strand
  misc_feature 20..21: note = n=dT
ttgaaggtgctgctgctgcnn

SEQ ID NO: 5 - RNA, 21 nt, synthetic construct
  source 1..21: mol_type = other RNA
  misc_feature 1..21: note = hs-EGR1-si-3 sense strand
  misc_feature 20..21: note = n=dT
ggacaagaaagcagacaaann

SEQ ID NO: 6 - RNA, 21 nt, synthetic construct
  source 1..21: mol_type = other RNA
  misc_feature 1..21: note = hs-EGR1-si-3 antisense strand
  misc_feature 20..21: note = n=dT
tttgtctgctttcttgtccnn

SEQ ID NO: 7 - RNA, 23 nt, synthetic construct
  source 1..23: mol_type = other RNA
  misc_feature 1..23: note = hs-COL10A1-si-1 sense strand
  misc_feature 22..23: note = n=dT
ccctacaccataaagagtaaann

SEQ ID NO: 8 - RNA, 23 nt, synthetic construct
  source 1..23: mol_type = other RNA
  misc_feature 1..23: note = hs-COL10A1-si-1 antisense strand
  misc_feature 22..23: note = n=dT
tttactctttatggtgtagggnn

SEQ ID NO: 9 - RNA, 23 nt, synthetic construct
  source 1..23: mol_type = other RNA
  misc_feature 1..23: note = hs-COL10A1-si-2 sense strand
  misc_feature 22..23: note = n=dT
cctgtaatgtacacctatgatnn

SEQ ID NO: 10 - RNA, 23 nt, synthetic construct
  source 1..23: mol_type = other RNA
  misc_feature 1..23: note = hs-COL10A1-si-2 antisense strand
  misc_feature 22..23: note = n=dT
atcataggtgtacattacaggnn

SEQ ID NO: 11 - RNA, 23 nt, synthetic construct
  source 1..23: mol_type = other RNA
  misc_feature 1..23: note = hs-COL10A1-si-3 sense strand
  misc_feature 22..23: note = n=dT
gcatcaaaggtgatagaggttnn

SEQ ID NO: 12 - RNA, 23 nt, synthetic construct
  source 1..23: mol_type = other RNA
  misc_feature 1..23: note = hs-COL10A1-si-3 antisense strand
  misc_feature 22..23: note = n=dT
aacctctatcacctttgatgcnn

SEQ ID NO: 13 - RNA, 21 nt, synthetic construct
  source 1..21: mol_type = other RNA
  misc_feature 1..21: note = hsa-miR-218-5p
ttgtgcttgatctaaccatgt

SEQ ID NO: 14 - RNA, 22 nt, synthetic construct
  source 1..22: mol_type = other RNA
  misc_feature 1..22: note = hsa-miR-218-1-3p
atggttccgtcaagcaccatgg